(19) United States
(12) Patent Application Publication
(10) Pub. No.: US 2002/0115613 A1
(43) Pub. Date: Aug. 22, 2002

(54) Treatment of prostate cancer

(76) Inventor: M. Vijay Kumar, Martinez, GA (US)

Correspondence Address: Cynthia B. Rothschild, Kilpatrick Stockton LLP, 1001 West Fourth Street, Winston-Salem, NC, 27101, US

(21) Appl. No.: 10/077,435

(22) Filed: Feb. 15, 2002

Related U.S. Application Data
(60) Provisional application No. 60/269,698, filed on Feb. 16, 2001.

(51) Int. Cl. A61K 38/17; A61K 31/56

(52) U.S. Cl. 514/12; 514/179

(57) ABSTRACT

The present invention provides methods and compositions for treating cancer, and even more preferably, prostate cancer. In one aspect, the present invention comprises a method for inducing cell death in cancer cells comprising treating at least a portion of the cancer cells with an effective amount of TRAIL and an effective amount of an antiprogestin sufficient to induce apoptosis in at least a portion of the treated cancer cells. In another aspect, the present invention comprises a composition for treating cancer by inducing cell death in cancer cells comprising a pharmaceutical composition comprising an effective amount of TRAIL and an effective amount of an antiprogestin sufficient to induce apoptosis in at least a portion of the cancer cells exposed to the composition. In an embodiment, the antiprogestin is Mifepristone.

This application claims priority to provisional application Serial No. 60/269,698 filed Feb. 16, 2001. Provisional Application Serial No. 60/269,698 is incorporated in its entirety by reference herein.

### FIELD OF THE INVENTION

The present invention relates to methods and compositions for treating cancer. In particular, the present invention relates to therapies for treating prostate cancer, and even more specifically, to therapies for inducing apoptosis in prostate cancer cells. In an embodiment of the present invention, a combination of TRAIL (Tumor necrosis factor α—Related Apoptosis Inducing Ligand) and an antiprogestin, such as Mifepristone, are utilized to induce apoptosis in prostate cancer cells.

### BACKGROUND

Prostate cancer is one of the most commonly diagnosed cancers and a leading cause of cancer-related death among American men. Prostate cancer is a multi-focal disease with clones of androgen-sensitive and androgen-refractory cells (N. Kyprianou and J. Isaacs, Biochem. Biophys. Res. Comm., 165, 73-81, 1989; Kyprianou, N., et al., World J. Urol., 12, 299-303, 1994; M. Tenniswood and H. Michna, Ernst Schering Research Foundation Workshop, 14, Springer-Verlag, Berlin Heidelberg, 1995). The role androgen receptors (AR) may play in prostate cancer is not clear. Although androgen depletion therapy results in regression of the tumor, it returns as an androgen-refractory cancer. Some androgen-refractory tumors express increased levels of androgen receptors, suggesting that continued proliferation of androgen-refractory prostate cells may be influenced by androgens (Linja, M. J., et al., Cancer Res., 61, 3350-3555, 2001). Paradoxically, increased expression of AR may also be responsible for inhibition of growth and may induce apoptosis (Joly-Pharaboz, M. O., et al., J. Steroid Biochem. Mol. Biol., 55, 67-76, 1995; Joly-Pharaboz, M. O., et al., J. Steroid Biochem. Mol. Biol., 73, 237-249, 2000; Dai, J. L., et al., Steroids, 61, 531-539, 1996; Umekita, Y., et al., Proc. Natl. Acad. Sci., USA, 93, 11802-11807, 1996; Zhau, H. Y. E., et al., Proc. Natl., Acad. Sci., USA, 93, 15152-15157, 1996; Heisler L. E., et al., Mol. Cell. Endocr., 126, 59-73, 1997; Shen, R., et al., Endocrinology, 141, 1699-1704, 2000).

Apoptosis is a term used to describe a series of cellular events which occur to bring about cell death. As apoptosis is inhibited in cancer, induction of apoptosis is an option for treatment of cancer. For example, prostate cancer cells are known to exhibit differing sensitivities to various anti-cancer agents depending upon cell type, and may undergo apoptosis utilizing one or more pathways involving Bcl₂, Fas:FasL, TNFα, TGFβ and p53. Even androgen-refractory prostate cancer cells retain the capacity to undergo apoptotic cell death (Denmeade, S. R., et al., Prostate, 39, 269-279, 1999; Wang, J-D., et al., Prostate 40, 50-55, 1999; Marcelli, M., et al., Prostate, 42, 260-273, 2000). In prostate cancer cells, apoptosis has been induced by a variety of agents such as staurosporine (Zhang, H., et al., Prostate, 29, 69-76, 1996; Marcelli, M., et al., Cancer Res., 59, 398-406, 1999; Li, X., et al., Cancer Res., 61, 1699-2706, 2001), levostatin (Marcelli, M., et al., Cancer Res., 58, 76-83, 1998), thapsigargin (Denmeade, S. R., et al., Prostate, 39, 269-279, 1999), okadaic acid (Bowen, C., et al., Cell Death Diff., 6, 394-401, 1999), camptothecin (Wang, J-D., et al., Prostate 40, 50-55, 1999), Mifepristone (El Etreby, M. F., et al., Prostate 43, 31-42, 2000; Sridhar, S., et al., Cancer Res., 61, 7179-7183, 2001) and TRAIL (van Ophoven, A., et al., Prostate Cancer Prostatic Dis., 2, 227-233, 1999; Yu, R., et al., Cancer Res., 60, 2384-2389, 2000; Nesterov, A., et al., J. Biol. Chem., 276, 10767-10774, 2001).

TRAIL is a recent addition to the tumor necrosis factor α family of apoptic inducing agents. TRAIL has sequence similarities to TNF α and to Fas-ligand (Yeh, W-C., et al., Immunol. Rev., 169, 283-302, 1999; Pitti, R. M., et al., J. Biol. Chem., 271, 12687-12690, 1996; Ashkenazi, A., et al., Science, 281, 1305-1308, 1998), and induces apoptosis through its interaction with the death domain receptors, DR4 (TRAIL-R1) (Pan, G., et al., Science 276, 111-113, 1997) and DR5 (TRAIL-R2, TRICK2 or KILLER) (Pan, G., et al., Science, 277, 815-818, 1997; Sheridan, J. P., et al., Science 277, 818-821, 1997; Walczak, H., et al., EMBO J., 16, 5386-5397, 1997). Certain cancer cells have been shown to be sensitive to the ability of TRAIL to induce apoptosis. The combination of TRAIL with other apototic agents such as etoposide significantly increased apoptosis in breast cancer, kidney cells and glioblastoma (Marsters, S. A., et al., Curr. Biol., 6, 750-752, 1996; Gibson, S. B., et al., Mol. Cell. Biol., 20, 205-212, 2000). The reasons for the sensitivity of certain cancer cells to TRAIL is not clear, and may include differences in TRAIL receptors (or decoy receptors) or differential activation of downstream pathways. Early studies have shown that not all prostate cancer cells are sensitive to TRAIL treatment.

An effective treatment for prostrate cancer will kill both androgen-responsive and androgen-refractory cancer cells. Whereas commonly used androgen deprivation therapies induce apoptotic cell death in androgen-sensitive cells (Colombel, M. C., et al., Methods Cell. Biol., 46, 27-34, 1995; Buttyan, R., et al., In: Prostate—Basic and Clinical Aspects., pp 201-218, Naz R K (ed), CRC Press, Boca Raton, 1997; Perlman, H., et al., Cell Death Differentiation 6, 48-54, 1999; Bruckheimer, E. M., et al., Sem. Oncol., 26, 382-398, 1999), effective chemotherapy for androgen-refractory cancer is not available (Kozlowski, J., et al., Urol. Clin. N. Am., 18, 15-24, 1991; Santen, R. J., J. Clin. Endocrinol. Metab., 75, 685-689, 1992; Kreis, W., Cancer Invest., 13, 296-312, 1995). It would be advantageous to have means of inducing apoptosis in all prostate cancer cell types, including both androgen-sensitive and androgen-refractory prostate cancer cells.

### SUMMARY OF THE INVENTION

The present invention is concerned with the treatment of cancer by inducing apoptosis, or programmed cell death, in cancer cells. More specifically, the present invention is concerned with treating prostate cancer by inducing apoptosis in both androgen-sensitive and androgen-insensitve cells (which are generally refractive to conventional means of therapy, such as androgen depletion). Thus, the present invention provides a means to reduce the population of cancer cells in a tumor. The present invention may be used alone, or in combination with other methods of treatment such as surgery, radiation therapy, or other types of chemotherapy.

In one aspect, the present invention comprises a method for inducing cell death in cancer cells, the method comprising treating cancer cells with an effective amount of TRAIL sufficient to induce apoptosis in at least a portion of the treated cancer cells.

In another aspect, the present invention comprises a method for inducing cell death in cancer cells, the method comprising treating cancer cells with an effective amount of TRAIL and an effective amount of an antiprogestin sufficient to induce apoptosis in at least a portion of the treated cancer cells.

In another aspect, the present invention comprises a method for treating cancer by inducing cell death in cancer cells, the method comprising treating cancer cells with a pharmaceutical composition comprising an effective amount of TRAIL and an effective amount of Mifepristone sufficient to induce apoptosis in at least a portion of the treated cancer cells.

In yet another aspect, the present invention also comprises a composition for treating cancer by inducing cell death in cancer cells comprising an effective amount of TRAIL in a pharmaceutical carrier, wherein an effective amount comprises sufficient TRAIL to induce apoptosis in at least a portion of the cancer cells exposed to the composition of the present invention.

Also, the present invention comprises a composition for treating cancer by inducing cell death in cancer cells comprising an effective amount of TRAIL and an antiprogestin in a pharmaceutical carrier, wherein an effective amount comprises sufficient TRAIL and antiprogestin sufficient to induce apoptosis in at least a portion of the cancer cells exposed to the composition of the present invention.

The present invention also comprises a composition for treating cancer by inducing cell death in cancer cells comprising an effective amount of TRAIL and Mifepristone in a pharmaceutical carrier, wherein an effective amount comprises sufficient TRAIL and Mifepristone sufficient to induce apoptosis in at least a portion of cancer cells exposed to the composition.

In addition, the present invention comprises kit for pharmaceutical treatment of cancer comprising: (a) a pharmacologically effective amount of TRAIL packaged in a sterile container; (b) a pharmacologically effective amount of an antiprogestin packaged in a sterile container; (c) at least one aliquot of a pharmaceutical carrier; and (d) instructions for application of the TRAIL and antiprogestin to a patient having cancer.

The foregoing focuses on the more important features of the invention in order that the detailed description which follows may be better understood and in order that the present contribution to the art may be better appreciated. There are, of course, additional features of the invention which will be described hereinafter and which will form the subject matter of the claims appended hereto. It is to be understood that the invention is not limited in its application to the specific details as set forth in the following description and figures. The invention is capable of other embodiments and of being practiced or carried out in various ways.

From the foregoing summary, it is apparent that an object of the present invention is to provide methods and compositions for inducing apoptosis in cancer, and more specifically, in both androgen insensitive and androgen responsive prostate cancer cells. These, together with other objects of the present invention, along with various features of novelty which characterize the invention, are pointed out with particularity in the claims and description provided herein.

### BRIEF DESCRIPTION OF THE FIGURES

FIG. 1 shows that pre-treatment with Mifepristone facilitates TRAIL-induced apoptosis in prostate cancer cells in accordance with an embodiment of the present invention. LNCaP (androgen responsive) (A) and (C) or LNCaP C4-2 (androgen insensitive) (B) and (D) cells were treated with 10 μM Mifepristone (Mif) for three days and/or treated (without washing) with TRAIL for the indicated time periods. The effects on cells were assayed using MTT (A) and (B) or Apoptosense (C) and (D) assays. Panel (E) shows that the response of PC3Neo and PC3AR to TRAIL is not mediated through the androgen receptor. Androgen-responsive (PC3AR) and androgen-insensitive (PC3Neo) cells were treated with 400 ng/ml TRAIL (T) for indicated periods or pretreated with the anti-androgen, 5 uM hydroxyflutamide anti-androgen (F), and then with 400 ng/ml TRAIL for indicated periods.

FIG. 2 shows expression of death and decoy receptors in response to TRAIL and Mifepristone in accordance with an embodiment of the present invention. LNCaP (A) and LNCaP C4-2 (B) cells were treated with Mifepristone (for 3 days) and/or TRAIL for 2 and 4 hours (hr), wherein, Control=no treatment; Mif=Mifepristone treated; TRAIL=TRAIL treated; and Mif+TRAIL=TRAIL and Mifepristone treated. In panel (C), PC3Neo and PC3AR cells were treated with 400 ng/ml TRAIL. C denotes control and T denotes TRAIL treatment. Actin 1 refers to loading control for DR5 and DR4 blots, while Actin 2 refers to DcR1 and DcR2 blots.

FIG. 3 shows activation of caspases and truncation of Bid in accordance with an embodiment of the present invention wherein (A) and (B) show activation of procaspase-8 and truncation of Bid in response to TRAIL and Mifepristone, and (C) and (D) show activation of procaspase-3 and -7 in response to TRAIL and Mifepristone. Panels (A) and (B) show LNCaP cells (A) and LNCaP C4-2 cells (B), respectively, treated with Mifepristone (for 3 days) and/or TRAIL for 2, 4, 6, 8, 16 or 20 hours (hr): 1=controls; 2=Mifepristone treated; 3=TRAIL treated; and 4=TRAIL and Mifepristone treated. Identified are the 57KDa procaspase-8 (PC-8), intermediate 43KDa product and activated caspase-8 at 18 KDa (Cl 8), Bid, and truncated Bid (tBID). Panels (C) and (D) show LNCaP (C) and LNCaPC4-2 (D) cells, respectively, pre-treated with Mifepristone and/or with TRAIL for 2, 4, 6, 8, 16 or 20 hour and detection of procaspase-3 and 7 (PC 3; PC 7) as well as cleaved products (Cl 3; Cl 7), wherein 1=controls, 2=Mifepristone treated, 3=TRAIL treated, and 4=TRAIL and Mifepristone treated

FIG. 4 shows that caspase-9 is activated by treatment of prostate cancer cells in accordance with an embodiment of the present invention, wherein LNCaP (A) and LNCaP C4-2 (B) cells, respectively, were treated as indicated and caspase-9 activity was determined using calorimetric assays. Values are expressed as mean (±SE) caspase activity in units/μg protein (n=4).

FIG. 5 shows that cytochrome c is released in response to treatment of prostate cancer cells with Mifepristone (M) or vehicle for three days and then (without washing) TRAIL (T) for the indicated time periods (in minutes) in accordance with an embodiment of the present invention wherein release of cytochrome c (Cyto c) into the cytoplasm was measured by immunoblot for LNCaP cells (A) and LNCaP C4-2 cells (B).

FIG. 6 shows that over-expression of IκB affected NFκB-mediated responses to TRAIL in accordance with an embodiment of the present invention. In panel (A), cells were infected with IκBM adenoviral construct for 3 hr before treatment with 400 ng/ml TRAIL for 20 hr and cell survival assayed. C denotes controls, T is TRAIL treated and IκB denotes cells infected with viral construct and treated with TRAIL. In panel (B), PC3Neo (Neo) and PC3AR (AR) cells were infected and treated as described in (A). Proteins were extracted and analyzed. (+) denotes infection with IκBM construct and (−) denotes uninfected controls. Panel (C) illustrates that disruption of NFκB function altered response of apoptotic members, wherein cells were treated for indicated periods with or without infection with IκBM construct, and C denotes control and T denotes TRAIL treatment; other abbreviations are as for FIG. 3.

FIG. 7 shows the effect of inhibitors on specific caspases during the apoptic response in accordance with an embodiment of the present invention. Cells were treated with drugs in the presence or absence of inhibitors for caspases-8 (Z-IETD-FMK) or -9 (Z-LEHD-FMK). Abbreviations are as described in earlier figures.

FIG. 8 shows that the reduced apoptic response of LNCaP cells (compared to LNCaP C4-2 cells) to TRAIL is not mediated through Akt. Cells were treated with TRAIL alone for the indicated time periods or pretreated for 3 days with Mifepristone and then treated with TRAIL for different durations and the cytosol fraction was examined for the levels of phosphorylated Akt (pAkt).

### DETAILED DESCRIPTION OF THE INVENTION

Prostate cancer is a leading cause of cancer-related death in American men. Although androgen depletion therapy results in regression of the tumor, the cancer often returns as an androgen-refractory cancer. The present invention describes therapies and compositions for treating cancer, and even more specifically, to treatment of prostate cancer by inducing apoptosis in both androgen-responsive and androgen-resistant prostate cancer cells.

In one aspect, the present invention comprises a method for inducing cell death in cancer cells, the method comprising treating cancer cells with an effective amount of TRAIL sufficient to induce apoptosis in at least a portion of the treated cancer cells.

Preferably, the dose of TRAIL in the pharmaceutical composition results in a local concentration of TRAIL at the tumor which ranges from 1 to 1,000 ng/ml. More preferably, the dose of TRAIL in the pharmaceutical composition results in a local concentration of TRAIL at the tumor which ranges from 200 to 600 ng/ml. Even more preferably, the dose of TRAIL in the pharmaceutical composition results in a local concentration of TRAIL at the tumor which ranges from 350 to 450 ng/ml.

In an embodiment, the cancer cells comprise prostate cancer cells. Preferably, the cancer cells comprise androgen responsive cells. Also preferably, the cancer cells also comprise androgen insensitive cells. The cancer cells may also comprise other types of cancer such as breast and colon cancer.

In an embodiment, the treatment of cells with TRAIL is associated with an increase in at least one death receptor. Preferably, the death receptor increased by treatment of cells with TRAIL is DR4 and/or DR5.

In an embodiment, treatment of cells with TRAIL is associated with an increase in the amount of activated caspase enzymes in at least a portion of the treated cells. Preferably, the caspases which are activated upon exposure of cells to TRAIL comprise caspase-8, caspase-3, caspase-9, or caspase-7. Also, in an embodiment, the treatment of cancer cells with TRAIL is associated with an increase in truncated BID protein (tBid) in at least a portion of the treated cells.

Preferably, treatment of cancer cells with TRAIL is associated with a decrease in mitochondrial function. Also preferably, treatment of cancer cells with TRAIL is associated with an increase in apoptosome formation.

To increase the efficacy of the treatment, the cancer cells may be treated with a compound which reduces the concentration of active NFκB in the nucleus of the treated cells. In an embodiment, the compound which reduces the concentration of active NFκB comprises IκB or an analogue thereof, wherein the analogue of IκB comprises a protein which prevents activation of NFκB. Alternatively an NFκB transcription factor decoy may be employed.

Preferably, the treatment comprises intravenous administration of the pharmaceutical composition of the present invention. Also preferably treatment is used in combination with other means of treatment such as surgery, chemotherapy, or radiation therapy.

In another aspect, the present invention comprises a method for inducing cell death in cancer cells, the method comprising treating cancer cells with an effective amount of TRAIL and an effective amount of an antiprogestin sufficient to induce apoptosis in at least a portion of the treated cancer cells. In a preferred embodiment, the antiprogestin comprises Mifepristone. In an embodiment, the cancer cells are treated with the antiprogestin prior to being treated with TRAIL. Alternatively, the cancer cells may be treated with the antiprogestin and TRAIL substantially concurrently.

Preferably, the dose of TRAIL in the pharmaceutical composition results in a local concentration of TRAIL at the tumor which ranges from 1 to 1,000 ng/ml. More preferably, the dose of TRAIL in the pharmaceutical composition results in a local concentration of TRAIL at the tumor which ranges from 200 to 600 ng/ml. Even more preferably, the dose of TRAIL in the pharmaceutical composition results in a local concentration of TRAIL at the tumor which ranges from 350 to 450 ng/ml.

Preferably, the dose of the antiprogestin in the pharmaceutical composition results in a local concentration of the antiprogestin at the tumor which ranges from 1 to 1000 μM. More preferably, the dose of the antiprogestin in the pharmaceutical composition results in a local concentration of the antiprogestin at the tumor which ranges from 1 to 100 μM. Even more preferably, the dose of the antiprogestin in the pharmaceutical composition results in a local concentration of the antiprogestin at the tumor which ranges from 5 to 20 μM.

In an embodiment, the cancer cells comprise prostate cancer cells. Preferably, the cancer cells comprise androgen responsive cells. Also preferably, the cancer cells also comprise androgen insensitve cells. The cancer cells may also comprise other types of cancers such as breast and colon cancer. Generally, the method may be employed for cancers which have shown some sensitivity to either TRAIL and/or an antiprogestin.

In an embodiment, the treatment of cells with TRAIL and the antiprogestin is associated with an increase in at least one death receptor. Preferably, the death receptor increased by treatment of cells with TRAIL and the antiprogestin is DR4 and/or DR5.

In an embodiment, treatment of cells with TRAIL and the antiprogestin is associated with an increase in the amount of activated caspase enzymes in at least a portion of the treated cells. Preferably, the caspases which are activated upon exposure of cells to TRAIL and the antiprogestin comprise caspase-8, caspase-3, caspase-9, or caspase-7. Also, in an embodiment, the treatment of cancer cells with TRAIL and the antiprogestin is associated with an increase in truncated BID protein (tBid) in at least a portion of the treated cells.

Preferably, treatment of cancer cells with TRAIL and the antiprogestin is associated with a decrease in mitochondrial function. Also preferably, treatment of cancer cells with TRAIL and the antiprogestin is associated with an increase in apoptosome formation.

To increase the efficacy of the treatment, the cancer cells may be treated with a compound which reduces the concentration of active NFκB in the nucleus of the treated cells. In an embodiment, the compound which reduces the concentration of active NFκB comprises IκB or an analogue thereof, wherein the analogue of IκB comprises a protein which prevents activation of NFκB. Alternatively an NFκB transcription factor decoy may be employed.

Preferably, the treatment comprises intravenous administration of the pharmaceutical composition of the present invention. Also preferably treatment is used in combination with other means of treatment such as surgery, chemotherapy, or radiation therapy.

In another aspect, the present invention comprises a method for treating cancer by inducing cell death in cancer cells, the method comprising treating cancer cells with a pharmaceutical composition comprising an effective amount of TRAIL and an effective amount of Mifepristone sufficient to induce apoptosis in at least a portion of the treated cancer cells.

In an embodiment, the cancer cells are treated with Mifepristone prior to being treated with TRAIL. Alternatively, the cancer cells may be treated with Mifepristone and TRAIL concurrently.

Preferably, the dose of TRAIL in the pharmaceutical composition results in a local concentration of TRAIL at the tumor which ranges from 1 to 1,000 ng/ml. More preferably, the dose of TRAIL in the pharmaceutical composition results in a local concentration of TRAIL at the tumor which ranges from 200 to 600 ng/ml. Even more preferably, the dose of TRAIL in the pharmaceutical composition results in a local concentration of TRAIL at the tumor which ranges from 350 to 450 ng/ml.

Preferably, the dose of Mifepristone in the pharmaceutical composition results in a local concentration of Mifepristone at the tumor which ranges from 1 to 1000 μM. More preferably, the dose of Mifepristone in the pharmaceutical composition results in a local concentration of Mifepristone at the tumor which ranges from 1 to 100 μM. Even more preferably, the dose of Mifepristone in the pharmaceutical composition results in a local concentration of Mifepristone at the tumor which ranges from 5 to 20 μM.

In an embodiment, the cancer cells comprise prostate cancer cells. Preferably, the cancer cells comprise androgen responsive cells. Also preferably, the cancer cells also comprise androgen insensitve cells. The cancer cells may also comprise other types of cancers such as breast and colon cancer. Generally, the method may be employed for cancers which have shown some sensitivity to either TRAIL and/or Mifepristone.

In an embodiment, the treatment of cells with TRAIL and Mifepristone is associated with an increase in at least one death receptor. Preferably, the death receptor increased by treatment of cells with TRAIL and Mifepristone is DR4 and/or DR5.

In an embodiment, treatment of cells with TRAIL and Mifepristone is associated with an increase in the amount of activated caspase enzymes in at least a portion of the treated cells. Preferably, the caspases which are activated upon exposure of cells to TRAIL and Mifepristone comprise caspase-8, caspase-3, caspase-9, or caspase-7. Also, in an embodiment, the treatment of cancer cells with TRAIL and Mifepristone is associated with an increase in truncated BID protein (tBid) in at least a portion of the treated cells.

Preferably, treatment of cancer cells with TRAIL and Mifepristone is associated with a decrease in mitochondrial function. Also preferably, treatment of cancer cells with TRAIL and Mifepristone is associated with an increase in apoptosome formation.

To increase the efficacy of the treatment, the cancer cells may be treated with a compound which reduces the concentration of active NFκB in the nucleus of the treated cells. In an embodiment, the compound which reduces the concentration of active NFκB comprises IκB or an analogue thereof, wherein the analogue of IκB comprises a protein which prevents activation of NFκB. Alternatively an NFκB transcription factor decoy may be employed.

Preferably, the treatment comprises intravenous administration of the pharmaceutical composition of the present invention. Also preferably treatment is used in combination with other means of treatment such as surgery, chemotherapy, or radiation therapy.

In another aspect, the present invention also comprises a composition for treating cancer by inducing cell death in cancer cells comprising an effective amount of TRAIL in a pharmaceutical carrier, wherein an effective amount comprises sufficient TRAIL to induce apoptosis in at least a portion of cancer cells exposed to said composition.

Preferably, the dose of TRAIL results in a local concentration of TRAIL at the tumor which ranges from 1 to 1,000 ng/ml. More preferably, the dose of TRAIL results in a local concentration of TRAIL at the tumor which ranges from 200 to 600 ng/ml. Even more preferably, the dose of TRAIL results in a local concentration of TRAIL at the tumor which ranges from 350 to 450 ng/ml.

In an embodiment, the cancer cells comprise prostate cancer cells. Preferably, the cancer cells comprise androgen responsive cells. Also preferably, the cancer cells also comprise androgen insensitve cells. The cancer cells may also comprise other types of cancers such as breast and colon cancer.

In an embodiment, treatment of cells with the composition is associated with an increase in at least one death receptor. Preferably the death receptor induced by treatment with TRAIL is DR4 and/or DR5.

In an embodiment, treatment of cells with the composition of the present invention is associated with an increase in the amount of activated caspase enzymes in at least a portion of the treated cells. Preferably, the caspases which are activated comprise caspase-8, caspase-3, caspase-9, or caspase-7. In an embodiment, the treatment of cancer cells with TRAIL is associated with an increase in truncated BID protein (tBid) in at least a portion of the treated cells.

Preferably, treatment of cancer cells with TRAIL is associated with a decrease in mitochondrial function. Also preferably, treatment of cancer cells with TRAIL is associated with an increase in apoptosome formation.

To increase the efficacy of the treatment, the composition may include a compound which reduces the concentration of active NFκB in said cells. In an embodiment, the compound which reduces the concentration of active NFκB comprises IκB or a structural analogue thereof, wherein the structural analogue of Iκβ comprises a protein which prevents activation of NFκB. Alternatively an NFκB transcription factor decoy may be employed.

The present invention also comprises a composition for treating cancer by inducing cell death in cancer cells comprising an effective amount of TRAIL and an antiprogestin in a pharmaceutical carrier, wherein an effective amount comprises sufficient TRAIL and antiprogestin sufficient to induce apoptosis in at least a portion of the cancer cells exposed to the composition. In an embodiment, the antiprogestin is Mifepristone.

In an embodiment, the antiprogestin and TRAIL are packaged in such a manner that the antiprogestin is at least partially released for application to the cancer prior to the release of the TRAIL. Alternatively, the antiprogestin and TRAIL may be packaged in such a manner so as to be released substantially simultaneously.

Preferably, the dose of TRAIL results in a local concentration of TRAIL at the tumor which ranges from 1 to 1,000 ng/ml. More preferably, the dose of TRAIL results in a local concentration of TRAIL at the tumor which ranges from 200 to 600 ng/ml. Even more preferably, the dose of TRAIL results in a local concentration of TRAIL at the tumor which ranges from 350 to 450 ng/ml.

Preferably, the dose of antiprogestin in the pharmaceutical composition results in a local concentration of antiprogestin at the tumor which ranges from 1 to 1,000 μM. More preferably, the dose of antiprogestin in the pharmaceutical composition results in a local concentration of antiprogestin at the tumor which ranges from 1 to 100 μM. Even more preferably, the dose of antiprogestin in the pharmaceutical composition results in a local concentration of antiprogestin at the tumor which ranges from 5 to 20 μM.

In an embodiment, the cancer cells comprise prostate cancer cells. Preferably, the cancer cells comprise androgen responsive cells. Also preferably, the cancer cells also comprise androgen insensitve cells. The cancer cells may also comprise other types of cancers such as breast and colon cancer. Generally, the method may be employed for cancers which have shown some sensitivity to either TRAIL and/or an antiprogestin.

In an embodiment, treatment of cells with the composition is associated with an increase in at least one death receptor. Preferably the death receptor induced by treatment with TRAIL and antiprogestin is DR4 and/or DR5.

In an embodiment, treatment of cells with the composition of the present invention is associated with an increase in the amount of activated caspase enzymes in at least a portion of the treated cells. Preferably, the caspases which are activated comprise caspase-8, caspase-3, caspase-9, or caspase-7. In an embodiment, the treatment of cancer cells with TRAIL and an antiprogestin is associated with an increase in truncated BID protein (tBid) in at least a portion of the treated cells.

Preferably, treatment of cancer cells with TRAIL and an antiprogestin is associated with a decrease in mitochondrial function. Also preferably, treatment of cancer cells with TRAIL and an antiprogestin is associated with an increase in apoptosome formation.

To increase the efficacy of the treatment, the composition may include a compound which reduces the concentration of active NFκB in said cells. In an embodiment, the compound which reduces the concentration of active NFκB comprises IκB or a structural analogue thereof, wherein the structural analogue of Iκβ comprises a protein which prevents activation of NFκB. Alternatively an NFκB transcription factor decoy may be employed.

The present invention also comprises a composition for treating cancer by inducing cell death in cancer cells comprising an effective amount of TRAIL and Mifepristone in a pharmaceutical carrier, wherein an effective amount comprises sufficient TRAIL and Mifepristone to induce apoptosis in at least a portion of cancer cells exposed to said composition.

In an embodiment, the Mifepristone and TRAIL are packaged in such a manner that the Mifepristone is at least partially released for application to the cancer prior to the release of the TRAIL. Alternatively, the Mifepristone and TRAIL may be packaged in such a manner so as to be released substantially simultaneously.

Preferably, the dose of TRAIL results in a local concentration of TRAIL at the tumor which ranges from 1 to 1,000 ng/ml. More preferably, the dose of TRAIL results in a local concentration of TRAIL at the tumor which ranges from 200 to 600 ng/ml. Even more preferably, the dose of TRAIL results in a local concentration of TRAIL at the tumor which ranges from 350 to 450 ng/ml.

Preferably, the dose of Mifepristone in the pharmaceutical composition results in a local concentration of Mifepristone at the tumor which ranges from 1 to 1,000 μM. More preferably, the dose of Mifepristone in the pharmaceutical composition results in a local concentration of Mifepristone at the tumor which ranges from 1 to 100 μM. Even more preferably, the dose of Mifepristone in the pharmaceutical composition results in a local concentration of Mifepristone at the tumor which ranges from 5 to 20 μM.

In an embodiment, the cancer cells comprise prostate cancer cells. Preferably, the cancer cells comprise androgen responsive cells. Also preferably, the cancer cells also comprise androgen insensitve cells. The cancer cells may also comprise other types of cancers such as breast and colon cancer. Generally, the method may be employed for cancers which have shown some sensitivity to either TRAIL and/or Mifepristone.

In an embodiment, treatment of cells with the composition of the present invention is associated with an increase in at least one death receptor. Preferably the death receptor induced by treatment with TRAIL and Mifepristone is DR4 and/or DR5.

In an embodiment, treatment of cells with the composition of the present invention is associated with an increase in the amount of activated caspase enzymes in at least a portion of the treated cells. Preferably, the caspases which are activated comprise caspase-8, caspase-3, caspase-9, or caspase-7. In an embodiment, treatment of cancer cells with TRAIL and Mifepristone is associated with an increase in truncated BID protein (tBid) in at least a portion of the treated cells.

Preferably, treatment of cancer cells with TRAIL and Mifepristone is associated with a decrease in mitochondrial function. Also preferably, treatment of cancer cells with TRAIL and Mifepristone is associated with an increase in apoptosome formation.

To increase the efficacy of the treatment, the composition may include a compound which reduces the concentration of active NFκB in said cells. In an embodiment, the compound which reduces the concentration of active NFκB comprises IκB or a structural analogue thereof, wherein the structural analogue of Iκβ comprises a protein which prevents activation of NFκB. Alternatively an NFκB transcription factor decoy may be employed.

In addition, the present invention comprises kit for pharmaceutical treatment of cancer comprising: (a) a pharmacologically effective amount of TRAIL packaged in a sterile container; (b) a pharmacologically effective amount of an antiprogestin packaged in a sterile container; (c) at least one aliquot of a pharmaceutical carrier; and (d) instructions for application of the TRAIL and antiprogestin to a patient having cancer. Preferably, the antiprogestin comprises Mifepristone. Also preferably, the cancer comprises prostate cancer.

TRAIL Induces Differential Apoptosis in Prostate Cells

Thus, in one aspect, the present invention comprises treatment of prostate cancer cells with TRAIL to induce apoptosis. Apoptosis describes the phenomenon of programmed cell death. Apoptosis is often required as a protective mechanism, in that programmed cell death occurs in those cells which may be deleterious to the organism. For example, apoptosis is induced in certain cells which are infected with a virus, for immune cells (e.g. CTLs) which are no longer required, cells with DNA damage, and cancer cells. Apoptosis may be triggered by intracellular or extracellular signals. The intracellular pathway occurs when intracellular damage in a cell causes the mitochondrial surface protein Bcl-2 to release Apaf-1, thus enabling cytochrome c to leak from the mitochondria. The released cytochrome c and Apaf-1 then bind to caspase-9 (a protease) to form apoptosomes which result in digestion of cellular proteins and DNA and the eventual phagocytosis of the cell. Extracellular mediation of apoptosis occurs via binding of ligands to death receptors and subsequent activation of caspase-8 (and other caspases), again leading to proteolysis and phagocytosis of the cells.

TRAIL (Tumor necrosis factor α—Related Apoptosis Inducing Ligand) is a recent addition to the tumor necrosis factor α family with sequence similarities to TNFα and to Fas-ligand (hence also called Apo2L). Although TRAIL may induce apoptosis in cancer cells, TRAIL does not appear to affect normal cells. TRAIL is a 281 amino acid type II transmembrane protein, which acts through specific receptors. TRAIL induces apoptosis through its interaction with the death domain receptors, DR4 (TRAIL-R1) and DR5 (TRAIL-R2, TRICK2 or KILLER). The function of death receptors is blocked by the expression of decoy receptors DcR1 (TRID/TRAIL-R3) (Sheridan, J. P., et al., Science 277, 818-821, 1997; Walczak, H., et al., EMBO J., 16, 5386-5397, 1997; Degli, et al., Immunity 7, 813-820, 1997) and DcR2 (TRUNDD/TRAIL-R4) (Degli et al., Immunity 7, 813-830, 1997). DR4, DR5, DcR1 and DcR2 are structurally related except that death receptors have a cytoplasmic death domain, where DcR1 and DcR2 lack this region.

In a preferred embodiment a combination of TRAIL and Mifepristone is employed. Antiprogestins, such as Mifepristone (RU 486) and onapristone, were developed for the inhibition of progesterone-dependent reproductive processes. In addition to their effects on reproductive functions, both Mifepristone and onapristone demonstrate anti-tumor activity in several hormone-dependent cancer models (M. F. El Etreby and Y. Liang, Breast Can. Res. Treat. 49, 109-117, 1998; El Etreby, M. F., et al., Breast Can. Res. Treat. 51, 149-168, 1998; Kamradt, M. C., et al., Gynecol., Oncol., 77, 177-182, 2000; Lucci A., et al., Intl. J. Oncol., 15, 541-546, 1999; Michna, H., et al., Breast Can. Res. Treat., 17, 155-156, 1990; Michna, H., et al., Breast Can. Res. Treat., 14, 275-288, 1989; Schneider, M. R., et al., Eur. J. Cancer Clin. Oncol., 25, 691-701, 1989; Schneider, M. R., et al., J. Steroid Biochem. Mol. Biol., 37, 783-787, 1990; Ekman, P., et al, Eur. J. Cancer 15, 257-262, 1979). For example, in cell culture and xenograft models, Mifepristone was shown to induce apoptosis in breast cancer, cervical carcinoma, endometrial cancer cells, lung cancer cells, meningioma and leiomyoma. Mifepristone can induce low levels of apoptosis in prostate cancer cells (El Etreby, M. F., et al., Prostate, 43, 31-42, 2000; Sridhar, S., et al., Cancer Res., 61, 7179-7183, 2001), and has been approved for clinical use in Europe and the United States for non-cancer related applications.

To achieve significant tumor regression, Mifepristone has been used in combination with other agents. For example, in cervical carcinoma, Mifepristone treatment along with radiation therapy induced apoptosis in radioresistant cells (Kamradt, M. C., et al., Gynecol. Oncol., 77, 177-182, 2000). Similarly, in breast cancer cells (MCF-7) rendered Adriamycin resistant, Mifepristone increased sensitivity to adriamycin in a dose-dependent manner (Lucci, A., et al., Int. J. Oncol., 15, 541-546, 1999). Another successful regimen for breast cancer treatment included a combination of Mifepristone and anti-estrogens such as Tamoxifen. Still, the mechnism by which Mifepristone enhances the effects of other agents is not clear. For example, in prostate cancer, Tamoxifen does not increase the efficacy of Mifepristone. Thus, Mifepristone and Tamoxifen each show a marginal ability to induce apoptosis in prostate cancer cells, and the effect of both agents together is the same as seen each agent alone (Sridhar et al., 2001).

The present invention provides a method for inducing apoptosis in prostate cancer cells regardless of whether the cells are resistant to androgens. The interaction between the androgen receptor and ability of prostate cells to respond to apoptic stimuli is not understood. For example, LNCaP prostate cancer cells are androgen-dependent, noninvasive prostate cancer cells. LNCaP C4-2 prostate cancer cells are derived from LNCaP and are more invasive and more metastatic than LNCaP. Although described as androgen-independent, LNCaP C4-2 cells have been shown to have 2-3 fold higher levels of androgen receptor than is found in LNCaP cells (Gregory et al., 2001).

Other prostate cancer cells lines have been derived to explore the function of the androgen receptor in prostate cancer. These cell lines include PC3Neo and PC3AR cell lines. Thus, PC3Neo cells are androgen-refractory PC3 cells stably transfected with a vector carrying the neomycin gene, and PC3AR cells are PC3 cells (originally androgen refractory) stably transfected with full length androgen receptor cDNA such that the PC3AR cells respond to androgen receptor binding (i.e. androgen-sensitive).

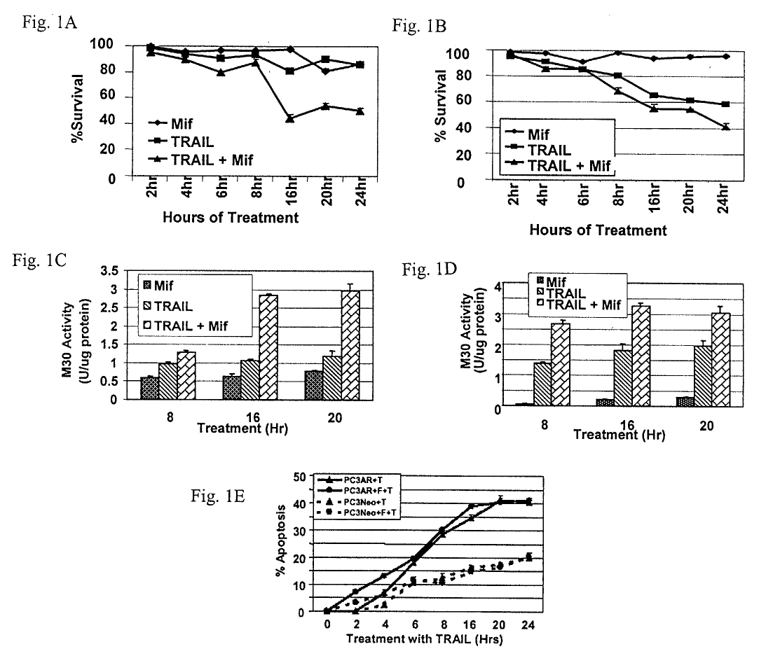

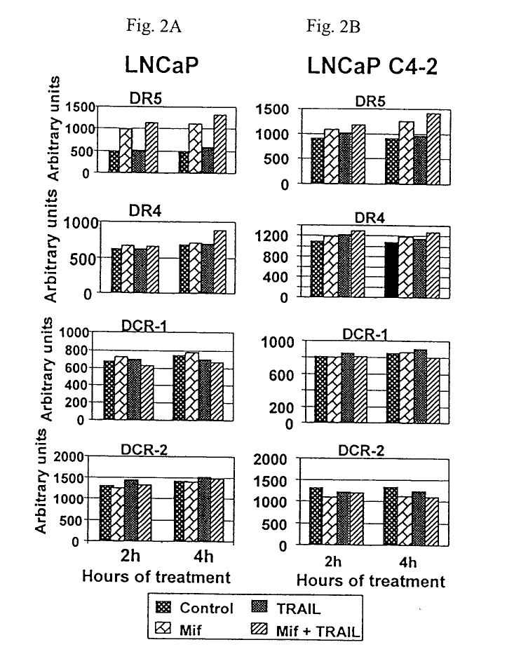

The present invention relies on the discovery that Mifepristone can increase the efficacy of TRAIL in inducing apoptosis in those prostate cancer cells which are resistant to the apoptic effects of TRAIL. Thus, as shown in FIG. 1 (Panels B and D), treatment of LNCaP C4-2 prostate cancer cells with 400 ng/ml TRAIL reduces cell survival as early as 8 hour and continues with exposure to the drug. Thus, LNCaP cells are relatively sensitive to the effects of TRAIL. In contrast, treatment of LNCaP cells with 400 ng/ml TRAIL does not alter cell survival significantly (FIG. 1, Panels A and C). Treatment of LNCaP cells with Mifepristone followed by TRAIL, however, results in a significant decrease in cell survival. Treatment of both LNCaP and LNCaP C4-2 cells with Mifepristone alone has little effect on survival (FIG. 1).

In an embodiment, the cancer cells are treated with the antiprogestin (e.g. Mifepristone) prior to being treated with TRAIL. In an embodiment, the cancer cells are treated with TRIAL prior to being treated with the antiprogestin. Alternatively, the cancer cells may be treated with the antiprogestin and TRAIL concurrently.

In an embodiment, there is an appropriate dose range for both Mifepristone and TRAIL. Thus, the dose of TRAIL preferably results in a local concentration of TRAIL at the tumor which ranges from 1 to 1,000 ng/ml. More preferably, the dose of TRAIL preferably results in a local concentration of TRAIL at the tumor which ranges from 200 to 600 ng/ml, and even more preferably, 350 to 450 ng/ml. Similarly, there is a preferred dose range for Mifepristone which ranges from 1 to 1,000 μM, or more preferably, 1 to 100 μM, and even more preferably, from 5 to 20 μM.

The presence or absence of functional androgen receptors is critical for designing treatment options for prostate cancer. The cell lines PC3Neo and PC3AR cell lines are useful for studying the effects androgen receptors have on prostate cancer cells. PC3AR cells are PC3Neo cells which have been engineered to express the androgen receptor. Preferably, androgen-insensitive (PC3Neo) and androgen-sensitive (or androgen-responsive) (PC3AR) cells treated with increasing concentrations of TRAIL exhibit dose-dependent and time-dependent increases in apoptosis (FIG 1E). In an embodiment, the preferential response of PC3AR cells to TRAIL is not mediated by the existence of an androgen receptor in these cells, but is due to other factors, as treatment with the anti-androgen, hydroxyflutamide (F) does not alter the response of these cell lines to TRAIL (FIG. 1E).

TRAIL+Mifepristone Causes An Increase In Death Receptors

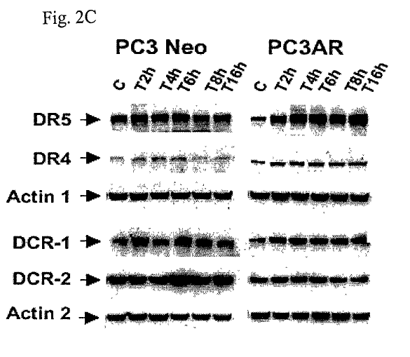

In an embodiment, the ability of Mifepristone to enhance the apoptic effects of TRAIL is mediated at least in part via an increase in the expression of death receptors. Treatment of LNCaP cells with Mifepristone results in a significant increase DR5 receptor expression (FIG. 2; see also, Sridhar, S., et al., Cancer Res., 61, 7179-7183, 2001), whereas treatment of LNCaP cells with TRAIL alone does not result in a significant increase in DR5 receptors (FIG. 2). Treatment of LNCaP cells with TRAIL and Mifepristone results in a greater increase in DR5 death receptors than with either agent alone (FIG. 2). Interestingly, LNCaP C4-2 cells exhibit higher basal levels of DR5, and therefore the up-regulation of DR5 with TRAIL and/or Mifepristone is not as significant as in LNCaP cells (FIG. 2; Sridhar et al., 2001).

In an embodiment, TRAIL also induces an increase in death receptors in PC3Neo and PC3AR cells. Thus, TRAIL significantly increases the expression of DR5 in both cell lines as early as 2 hr (FIG. 2C). In contrast, DR4 levels are induced by TRAIL in PC3AR cells, but not in PC3Neo cells, suggesting that increased apoptotic response of PC3AR cells may be due to higher response of DR5 and DR4. Also, the levels of the death receptor decoy DcR2 is significantly higher in PC3Neo cells compared to TRAIL-sensitive PC3AR cells (FIG. 2C), again suggesting that responses of prostate cancer cells may be regulated by death receptor levels.

Caspase-8 and Bid are Activated by TRAIL in Prostate Cancer Cells

Stimulation of death receptors (DR4 and DR5) activates caspase-8 (FLICE, MACH, Mch5), which forms the death inducing signaling complex (DISC) with death receptors and FADD (Fas-associating protein with a death domain). In type I apoptotic cells, caspase-8 propagates the death signal directly through the activation of procaspase-3. In type II cells, the apoptotic signal is amplified via the mitochondrial pathway, by truncation of Bid (a Bcl₂ family protein) to tBid, which translocates into the mitochondria and promotes release of cytochrome c leading to the formation of the apoptosome (Kaufman, S. H., et al., Bioessays, 22, 1007-1017, 2000).

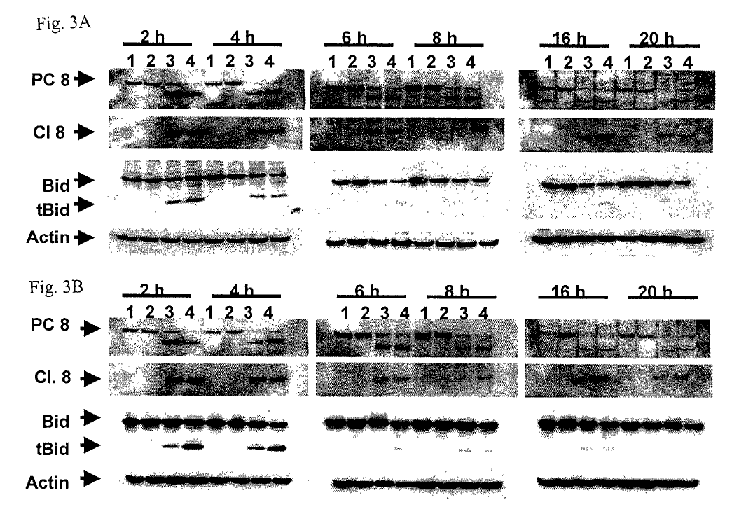

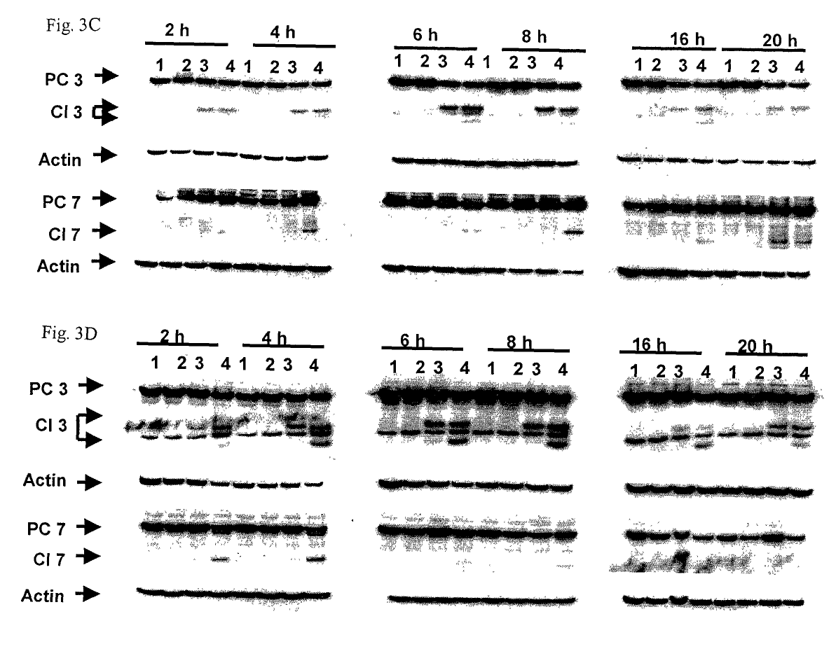

Thus, in an embodiment, TRAIL and Mifepristone act via caspase-8 to subsequently activate procapases 3, 9, and 7 to their activated forms. Preferably, treatment of prostate cancer cells with TRAIL (lanes 3) is accompanied by a decrease in the amount of 57 kDA procaspase-8 protein (PC 8), with concomitant appearance of two cleaved caspase-8 activated products of 46 kDa and 18 kDa (Cl 8) (FIG. 3). Similarly treatment of prostate cells with Mifepristone (lanes 2) is accompanied by an small increase in the activation of caspase-8 over controls (untreated cells) (lanes 1) (see also Sridhar et al., 2001, showing an increase in capase-8 activity in LNCaP and LNCaP C4-2 cells treated with Mifepristone). Although both TRAIL and Mifepristone can increase caspase-8 activation, in a preferred embodiment, treatment of prostate cancer cells with TRAIL and Mifepristone (lanes 4) is associated with increased activation of caspase-8 compared to the increase seen with either agent alone (FIG. 3). In a preferred embodiment, the ability of TRAIL and/or Mifepristone to increase caspase-8 is seen in many types of prostate cancer cells, both androgen responsive and androgen insensitive. Thus, the ability of TRAIL and/or Mifepristone to increase activation of caspase-8 is seen in TRAIL resistant (LNCaP and PC3Neo) and TRAIL-sensitive (LNCaP and PC3AR) cell lines.

Bid is a 22 kDa BH3 domain only, pro-apoptopic member of the Bcl2 family. Activated caspase-8 is responsible for the cleavage of Bid into a smaller NH₂-terminal and a larger COOH-terminal fragment (tBid). tBid is translocated into mitochondria, where it binds to either Bax or Bak, a necessary step for the release of cytochrome c into the cytoplasm (J. C. Martinou and D. R. Green, Nat. Rev. Mol. Cell Biol., 2, 63-66, 2001). Cytochrome c binds to Apaf1 in the presence of ATP to form the apoptosome with procaspase-9, which is responsible for the cascade of events resulting in cell death.

In an embodiment, treatment of prostate cancer cells with TRAIL or TRAIL and Mifepristone induces expression of tBid. For example, tBid is induced by treatment of LNCaP cells with TRAIL alone, or Mifepristone in combination with TRAIL (FIG. 3). tBid is also weakly induced by Mifepristone in LNCaP cells (Sridhar et al., 2001). In an embodiment, the amount of tBid seen with the combination of agents is greater than that seen with either agent alone (FIG. 3).

Activation of Caspases 3, 9, and 7 and Cytochrome c Release by TRAIL and Mifepristone

Caspase-3 may be activated via activated caspase-8, or indirectly, by caspase-9. Activation of caspase-3 yields two cleaved products: an initial 17 kDa protein and a mature 12 kDa protein.

In an embodiment, preferential activation of caspase-3 explains the ability of TRAIL (or TRAIL+Mifepristone) to induce apoptosis in a subset of prostate cancer cells (FIGS. 3C and 3D). Thus, treatment of LNCaP cells with Mifepristone (lanes 2) or TRAIL (lanes 3) activates caspase-3, but TRAIL+Mifepristone is required for full activation (FIG. 3C). In LNCaP C4-2 cells (FIG. 3D), Mifepristone is not required for induction of caspase-3, nor is it effective in inducing caspase-3.

Also preferably, treatment with TRAIL+Mifepristone induces activation of caspase-7. In an embodiment TRAIL+Mifepristone is more effective in activating caspase-7 than either agent alone in both androgen sensitive (LNCaP) and androgen insensitive cells (LNCaP C4-2) (FIGS. 3C and D).

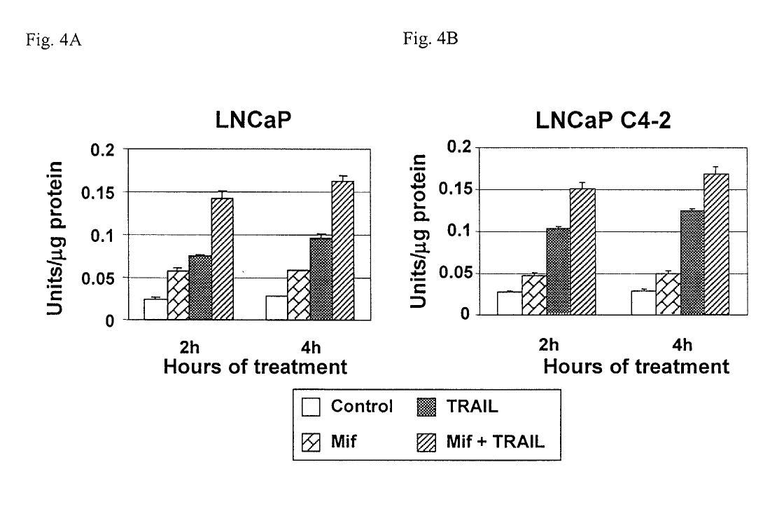

Also, as caspase-9 is a key protein in the formation of apoptosome, in an embodiment, treatment with TRAIL and/or Mifepristone induces caspase-9 activity. Thus, referring now to FIG. 4, treatment of cells with TRAIL, Mifepristone, or TRAIL and Mifepristone induces caspase-9 activity, with treatment with TRAIL and Mifepristone more effective than treatment with either agent alone.

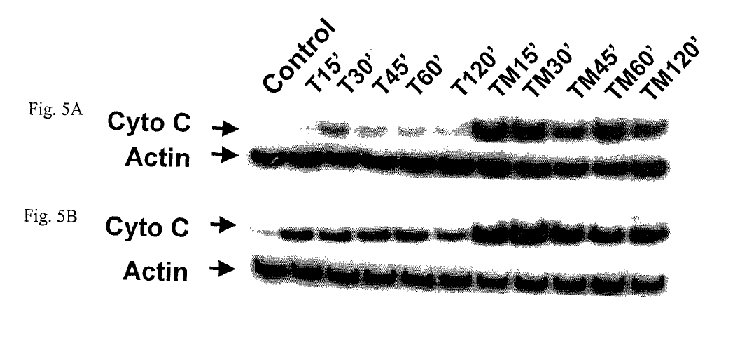

Apoptosis is associated with release of cytochrome c from the mitochondria. Cytochrome c binds to Apaf1 in the presence of ATP to form the aopotosome with procaspase-9, which is responsible for the cascade of events resulting in cell death. Thus, in an embodiment, treatment of prostate cells with TRAIL is associated with increase cytosolic cytochrome c (Cyto C), with TRAIL+Mifepristone required for high levels of cytosolic cytochrome in certain cells (e.g. LNCaP cells) but not others (e.g. LNCaP C42) (FIG. 5).

Increases in TRAIL Activity by NFkB Blockers

NFκB is an important member of survival pathway (M. W. Mayo and A. S. Baldwin, Biochim. Biophys. Acta, 1470, M55-M62, 2000). NFκB is involved in transformation and tumorigenesis and also suppresses apoptotic pathways. NFκB is sequestered in the cytoplasm in an inactive state by its interaction with IκB protein. Upon phosphorylation of Iκβ, followed by ubiquitination and degradation, NFκB translocates into the nucleus, where it induces transcriptional activity of target genes. NFκB can block TNFα- or TRAIL-induced apoptosis by influencing the function of DcR1, RIP (receptor interacting protein), FADD and caspase-8 (Wang, C-Y., et al, Science 281, 1680-1683, 1998; Sugiyama, H. J., et al., Biol. Chem., 274, 19532-19537, 1999; Hu, W-H., et al., J. Biol. Chem., 275, 10838-10844, 2000; Jones, D. R., et al., Ann. Thoracic Surg., 70, 930-937, 2000; K. Kuwano and N. Hara, Am. J. Respir. Cell Mol. Biol., 22, 147-149, 2000; Lin, Y., et al., Mol. Cell. Biol., 20, 6638-6645, 2000; Nagaki, M., et al., Hepatol., 32, 1272-1279, 2000; Bernard, D., et al., J. Biol. Chem., 276, 27322-27328, 2001). Furthermore, NFκB may indirectly affected apoptosis through Inhibitor of Apoptosis Proteins, cIAP1, cIAP2 and XIAP (Wang, C-Y., et al, Science 281, 1680-1683, 1998; Chu, Z-L., et al., Proc. Natl. Acad. Sci., USA, 94, 10057-10062, 1997; Van Atwerp, D. J., et al., Trends Cell Biol., 8, 107-111, 1998; Erl, W., et al., Circ. Res., 84, 668-677, 1999; M. Holcik and R. G. Korneluk, Nature Rev. Mol. Cell Biol, 2, 550-556, 2001; Levkau, G., et al., Circ. Res., 88, 282-290, 2001), which inhibit initiator and effector caspases (M. Holcik and R. G. Korneluk, Nature Rev., Mol. Cell Biol., 2, 550-556, 2001; Suzuki, Y., et al., J. Biol. Chem., 276, 27058-27063, 2001). NFκB also blocks apoptosis by increasing the expression of BCl_{XL}, an antiapoptotic protein (Ravi, R., Nature Cell Biol., 3, 409-416, 2001).

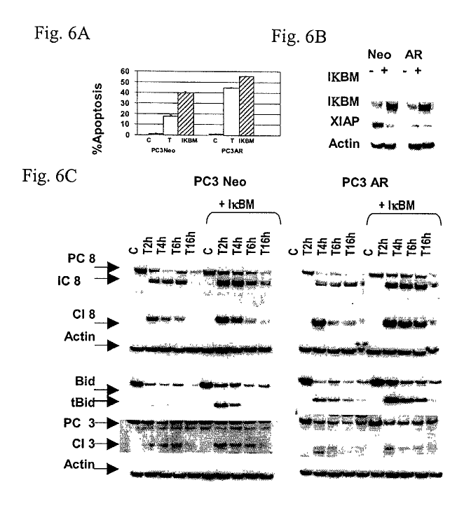

In an embodiment, the present invention further comprises treating prostate cancer cells with a compound which reduces the concentration of active NFκB in at least a portion of the treated cells. Preferably, the compound for reducing active NFκB comprises IκB or an analogue thereof. Thus, over-expressing mutated IκB (IκBM) which is resistant to cellular degradation reduces the amount of active NFκB in the cell, thereby sensitizing the cell to the apoptopic effects of TRAIL (T) (FIG. 6).

In an alternate embodiment, NFκB transcription factor decoys, which can be delivered intravenously and which block NFκB transcription, may be employed. Transcription factor decoys (TFDs) are single-stranded or double-stranded oligonucleotides that compete with endogenous cis DNA sequence elements in the regulatory regions of gene promoters for the binding of transcription factors. TFD oligodeoxyribonucleotides can bind to the transcription factor of interest and prevent the transcription factor from binding to its normal binding site. For example, NFκB TFDs can bind or “trap” NF-κB, and prevent transcription of genes activated by NFκB. Transcription factor decoys have been used successfully to block NFκB mediated gene transcription in endothelial cells and other inflammatory cells in vitro (Bielinska A. et al., Science 250, 997-999, 1990; Eck, S. L., et al., Mol. Cell. Biol., 13, 6530-6536, 1993; Goldring, C. E. P., et al., Biochem. Biophys. Res. Comm., 209, 73-79, 1995; and Morishita, R., et al., Nature Med., 3, 894-899, 1997).

Therapeutics

The invention contemplates methods of administration which are well known in the art. For example, in an embodiment, administration of the compound is intravenous. For example, in another embodiment, administration of the compound is intra-arterial. In yet another embodiment, administration of the compound is oral or as an aerosol. In another embodiment, administration of the compound is sublingual. In yet another embodiment, administration of the drug is transrectal, as by a suppository or the like.

Pharmaceutical formulations can be prepared by procedures known in the art. For example, the compounds can be formulated with common excipients, diluents, or carriers, and formed into tablets, capsules, suspensions, powders, and the like. Examples of excipients, diluents, and carriers, that are suitable for such formulations include the following: fillers and extenders such as starch, sugars, mannitol, and silicic derivates; binding agents such as carboxymethyl cellulose and other cellulose derivatives, alginates, gelatin, and polyvinyl pyrrolidone; moisturizing agents such as glycerol; disintegrating agents such as agar, calcium carbonate, and sodium bicarbonate; agents for retarding dissolution such as paraffin; resorption accelerators such as quaternary ammonium compounds; surface active agents such as cetyl alcohol, glycerol monostearate; adsorptive carriers such as kaolin and bentonite; and lubricants such as talc, calcium and magnesium stearate, and solid polyethyl glycols.

The compounds can also be formulated as elixirs or solutions for convenient oral administration or as solutions appropriate for parenteral administration, for instance by intramuscular, subcutaneous or intravenous routes. Additionally, the compounds are well suited to formulation as sustained release dosage forms and the like. The formulations can be so constituted that they release the active ingredient only or preferably in a particular part of the intestinal tract, possibly over a period of time. The coatings, envelopes, and protective matrices may be made, for example, from polymeric substances or waxes.

In addition, genetic constructs, such as transcription factor decoys (TFDs) or adenoviral construct comprising mutated IkBM as described herein (Example9) may be delivered into the cell by infection (e.g. as with recombinate adenovirus), direct transfection of naked (unprotected) DNA or may employ a type of carrier, such as liposomes. For example, in vivo delivery of TFDs using a hemaglutinating virus of Japan (HVJ)-liposome carrier (Morishita et al., Nature Med., 3: 894-899, 1997) or a Sendai virus-liposome carrier (U.S. Pat. No. 6,262,033) has been described in a mycocardial infarct model. Using HVJ liposomes, infusion of fluorescently labeled NF-κB TFDs into the left coronary artery resulted in fluorescence in coronary microvascular endothelial cells with a reduction of infarct size and reduced levels of IL-6 and VCAM mRNA. Recombinant adenoviruses have been shown to achieve high effecienty gene transfer after direct, in vivo delivery to airway epithelium pepatocytes, vascular endothelium, CNAS parenchyma and a number of other tissues (e.g. La Salle, Science 259, 988-990, 1993; Gomez-Foix, J. Biol. Chem., 267, 25129-25134, 1992; Rich, Human Gene Therapy, 4, 461-476, 1993; Guzman, Circulation Res., 73, 1201-1207, 1993; Bout, Human Gene Therapy, 5, 3-10, 1994).

As will be understood by those in the art, treatment with the compounds described herein may be varied as indicated by an individual's specific circumstances. Thus, in one aspect, the present invention comprises a composition for treating cancer by inducing cell death in cancer cells comprising an effective amount of TRAIL in a pharmaceutical carrier, wherein an effective amount comprises sufficient TRAIL to induce apoptosis in at least a portion of the cancer cells exposed to the composition of the present invention. In this aspect, the present invention relies on the discovery that TRAIL induces significant apoptosis in cancer cells via pathways involved in both extracellular induction (caspase-8) and intracellular induction (caspase-9).

Also, the present invention comprises a composition for treating cancer by inducing cell death in cancer cells comprising an effective amount of TRAIL and an antiprogestin, such as Mifepristone, in a pharmaceutical carrier, wherein an effective amount comprises sufficient TRAIL and antiprogestin to induce apoptosis in at least a portion of the cancer cells exposed to the composition of the present invention. In this aspect, the present invention relies on the discovery that antiprogestins, such as Mifepristone, induce significant apoptosis in cancer cells which are resistant to the apoptic effects of TRAIL.

In addition, the present invention comprises kit for pharmaceutical treatment of cancer comprising: (a) a pharmacologically effective amount of TRAIL packaged in a sterile container; (b) a pharmacologically effective amount of an antiprogestin packaged in a sterile container; (c) at least one aliquot of a pharmaceutical carrier; and (d) instructions for application of said TRAIL and said antiprogestin to a patient having cancer. In this aspect, the present invention relies on the understanding that in some cases pre-treatment with either TRAIL or the antiprogestin may increase the overall efficacy, whereas in some cases the compounds may be delivered substantially simultaneously.

### EXAMPLES

### Example 1

### Cell Culture and Treatment of Cells with TRAIL and Mifepristone

LNCaP and LNCaP C4-2 Cells

LNCaP were obtained from American Type Culture Collection (Rockville, Md.) and LNCaP C4-2 cells were purchased from Urocor Inc., Oklahoma City, Okla. Cells were grown in RPMI 1640 medium supplemented with 10% (LNCaP) or 5% (LNCaP C4-2) fetal bovine serum (Hyclone, Logan, Utah) and grown in the presence of 5% CO₂ at 37° C. Cells were treated with 10 μM Mifepristone (Sigma, St. Louis, Mo.) for three days or with increasing concentrations (from 200 ng/ml to 600 ng/ml) of TRAIL (Biomol Research Laboratories, Inc., Plymouth Meetings, Pa.) for varying time intervals 2, 4, 6, 8, 16, 20 and 24 hours (hr). Thus, for treatment with Mifepristone, cells were treated with Mifepristone for three days and then some cells treated (without washing) with TRAIL for indicated periods. Upon completion of the experiment, cells were harvested, and total proteins, cytosol or mitochondrial fractions were isolated as described below. At least three experiments were conducted with a minimum of four plates per treatment per experiment.

For experiments using inhibitors, the cells were pre-treated with increasing concentrations of the inhibitors (10 μM to 100 μM) for one hr and then with TRAIL. Caspase-8-specific (Z-IETD-FMK) and caspase-9-specific (Z-IEHD-FMK) inhibitors were purchased from Enzyme Systems Products, Livermore, Calif. The inhibitors were dissolved in DMSO; to limit cellular toxicity, care was taken not to exceed 0.2% DMSO in the culture medium. Appropriate controls included vehicle treated cells with or without the inhibitors.

PC3Neo and PC3AR Cells

Prostate cancer cell lines, PC3Neo and PC3AR were provided by Dr. K. Burnstein, University of Miami School of Medicine, Miami, Fla. PC3AR cells are a clonal cell line derived by stable transfection of PC3 with a full-length human AR cDNA (Dai, J. L., et al., Steroids, 61, 531-539, 1996). PC3Neo cells were stably transfected with neomycin vector alone. Cells were grown in RPMI 1640 with 5% FBS (Hyclone, Logan, Utah), 100 units/ml penicillin, 100 μg/ml streptomycin, 0.25 μg/ml fungizone and G418 (350 μg/ml) to select for neomycin-resistant cells. In experiments using antiandrogens, cells were maintained in 5% DCC stripped serum for at least 48 hr before treatment with hydroxyflutamide (Sigma Chemical Co., St. Louis, Mo.). The medium and all the drugs were replaced every two days. Cells were treated with 200 ng/ml, 400 ng/ml or 600 ng/ml of TRAIL (Biomol Research Laboratories, Inc, Plymouth Meeting, Pa.) for 2, 4, 6, 8, 16 and 20 hr. Cells were treated with 5 μM hydroxyflutamide for 5 days before the commencement of treatment with 400 ng/ml TRAIL.

### Example 2

### Apoptosis assays

Two methods were used to measure induction of apoptosis by drug treatment: the MTT assay and the Apoptosense assay. The MTT assay measures mitochondrial enzyme activity and is indicative of the number of surviving cells. The Apoptosense assay is an ELISA, which utilizes M30 antibody to recognize the C-terminal domain (amino acids 387-396) of cytokeratin 18 that is exposed in apoptotic cells after cleavage by caspases.

MTT Assay:

Cells were seeded in 96-well plates (6000 cells/well) and treatment was started 24 hr after seeding the cells. After completion of the treatment, cells were incubated at 37° C. with the MTT reagent for 3 hr and processed according to manufacturers instructions (Promega Corporation, Madison, Wis.). Color development was measured at 490 nm on a Spectra MAX 340 microplate reader (Molecular Devices, Menlo Park, Calif.).

Apoptosense Assay:

Cells were plated in petri dishes and after completion of the treatment, cells were harvested and total protein extracted as described below. Protein extract was added to 96-well plate coated with horseradish peroxidase-labeled mouse monoclonal M30 antibody (Peviva AB, Sweden), and a horseradish peroxide tracer solution was added to the wells and incubated for 4 hr. Color was developed by adding tetramethylbenzidine solution and the optical density of the bound antibody was determined at 450 nm on a Spectra MAX 340 microplate reader (Molecular Devices, Menlo Park, Calif.). Standard solution supplied by the supplier was used for generating standard curves.

### Example 3

### Western Blotting

Cells were harvested by trypsinization, washed in 1×PBS and cell pellets resuspended in lysis buffer (100 mM Tris-HCl pH 8.0, 0.1% Triton X 100 and protease inhibitor cocktail from Roche Diagnostic Corporation, Indianapolis, Ind.). Cells were incubated over ice for 30 min and centrifuged at 10,000 g at 4° C. for 10 min. The supernatant was collected and the protein concentration estimated using Bio-Rad protein reagent (Bio-Rad Laboratories, Hercules, Calif.).

Proteins (50 μg, unless stated otherwise) were separated on NuPAGE 10% Bis-Tris gels (Novex pre-cast mini gels, InVitrogen, Carlsbad, Calif.) at 100 volts for 1 hour in the presence of 1×MES-SDS running buffer (InVitrogen, Carlsbad, Calif.). Separated proteins were transferred to (PVDF) membranes (Bio-Rad Laboratories, Hercules, Calif.) at 42 volts for 2.5 hr using a Novex XCell II blotting apparatus in MES transfer buffer in the presence of NuPAGE antioxidant. Transfer of the proteins to the polyvinylidene difluoride (PVDF) membrane was confirmed by staining with Ponceau S (Sigma, St. Louis, Mo.). The blots were blocked in 5% non-fat dry milk in TBS, washed twice for 10 min each with TBS containing 0.1% Tween-20 and incubated for 2 hr at RT with primary antibody diluted in TBS containing 0.5% milk.

The following antibodies were used in the immunoblots: DR4, DR5 and cytochrome c (Imgenex, San Diego, Calif.), BID (BioSource International, Camarillo, Calif.), caspases-8, -9, and Akt (Cell Signaling Technology, Beverely, Mass.), caspase-3 (BD Pharmingen, San Diego, Calif.) Iκβ and XIAP (Cell Signaling Technology, Beverly, Mass.), cFLIP (StressGen Biotechnology Corp, Victoria, Canada), actin (Sigma Chemical Co., St. Louis, Mo.) and Cox II (Molecular Probes, Eugene, Oreg.). Immunoreactive bands were visualized using ECL detection system (Amersham, Pharmacia Biotech, Arlington Heights, Ill.) and signals were developed after exposure to X-ray film (X-Omat films, Eastman Kodak Company, Rochester, N.Y.).

### Example 4

### TRAIL Induced Differential Apoptosis in Prostate Cancer Cells: Pretreatment with Mifepristone Sensitized Cells to TRAIL

LNCaP and LNCaP C4-2, were treated with increasing concentrations (200 ng/ml, 400 ng/ml and 600 ng/ml) of TRAIL and the effects on cell survival and apoptosis were determined. Treatment of LNCaP cells with 400 ng/ml TRAIL did not alter cell survival significantly (FIG. 1A), while similar treatment of LNCaP C4-2 reduced cell survival as early as 8 hr and continued the trend through out the experiment (FIG. 1B). Treatment of LNCaP with Mifepristone followed by TRAIL resulted in significant decrease in survival with lowest values by 16 hr (FIG. 1A). Treatment of LNCaP C4-2 with Mifepristone and TRAIL decreased cell survival by 58% by 24 hr (FIG. 1B). Treatment of cells with Mifepristone alone during the same period showed little effect on survival, although survival was lower in LNCaP compared to LNCaP C4-2, which agreed with our earlier observations (Sridhar et al., 2001).

The above results were generated using the MTT assay, which measures mitochondrial enzyme activity and is a direct indicator of cell survival, not cell death. Values obtained using the MTT assay depend upon the number of cells present in the wells, which may vary with the ability of different cell lines to attach to the wells. Results were therefore confirmed using the Apoptosense assay. The Apoptosense assay is an ELISA-based assay which measures the cleavage of cytokeratin 18 in response to apoptosis-induced activation of caspases. Values obtained using this assay were expressed as antibody bound per unit protein concentration (instead of cell number). Treatment of LNCaP cells with TRAIL resulted in 0.9 to 1.2 U/μg M30-activity (FIG. 1C), whereas similar experiments with LNCaP C4-2 resulted in increasing M30 activity (from 1.4-2.0 U/ug) between 8 hr to 20 hr treatment (FIG. 1D). LNCaP cells treated with both Mifepristone and TRAIL showed 1.3 U/μg to 3.0 U/μg activity between 8 hr to 20 hr, while the activity was significantly higher in LNCaP C4-2 even by 8 hr. Mifepristone alone resulted in lower M30 activity in both cell lines although the LNCaP response was significantly higher than the LNCaP C4-2 response, confirming earlier results generated using other methods (Sridhar et al., 2001).

Experiments with lower concentrations of TRAIL (200 ng/ml) individually or with Mifepristone yielded lower cell death, whereas similar treatment with higher doses of TRAIL (600 ng/ml) did not increase apoptosis significantly (data not shown), suggesting that a threshold for induction of apoptosis was achieved by treating the cells with 400 ng/ml. Therefore, all further experiments were conducted by treating the cells with 400 ng/ml TRAIL.

The effect of TRAIL was also examined in two cells lines designed to test the effect of the androgen receptor on prostate cancer. Androgen-insensitive (PC3Neo) and androgen-responsive (PC3AR) cells (PC3 cells transfected with androgen receptor) were treated with increasing concentrations of TRAIL. TRAIL induced dose- and time-dependent increases in apoptosis in both cell lines. By 20 hr, 400 ng/ml TRAIL induced ˜41% apoptosis in PC3AR cells, whereas similar treatment of PC3Neo cells induce death only in ˜18% cells (FIG. 1E). Treatment of cells with 200 ng/ml TRAIL showed a lower response, although the trend was similar, while a higher dose (600 ng/ml) did not significantly increase apoptosis. These results demonstrate a differential response of PC3Neo and PC3AR cells to TRAIL treatment.

To determine whether the preferential response of PC3AR cells to TRAIL was mediated by the androgen receptor, PC3AR cells were treated with the anti-androgen, hydroxyflutamide (F) and then with TRAIL (T) (400 ng/ml). Pre-treatment with anti-androgen did not alter their response to TRAIL, suggesting that differences in response of PC3Neo and PC3AR to TRAIL is not mediated through the androgen receptor (FIG. 1E).

### Example 5

### Induction of Apoptosis is Associated with Up-Regulation of Death Receptors

These experiments determined the effect of TRAIL treatment on the expression of the death receptors DR5 and DR4, as well as the decoy receptors DcR1 and DCcR2. Comparative levels of proteins were measured by Western blotting as described in Example 3. Treatment of LNCaP with Mifepristone and TRAIL up-regulated the expression of DR5 significantly within 2 hr (FIG. 2A), with no significant increase with continued treatment. Expression of DR5 did not increase significantly when LNCaP were treated with TRAIL alone. Treatment of LNCaP C4-2 with Mifepristone and TRAIL showed a slight increase in DR5 (FIG. 2B). DR4 levels did not show significant changes in treated LNCaP and LNCaP C4-2 cells treated with TRAIL, Mifepristone, or TRAIL and Mifepristone (FIGS. 2A and B). Although the amounts of the decoy receptor DcR1 decreased slightly by 6-8 hr only in LNCaP C4-2 treated with TRAIL and Mifepristone, DcR2 did not change in either cell line.

Similar results were found using PC3Neo (androgen insensitve) and PC3AR (androgen responsive) cells. TRAIL significantly increased the expression of DR5 in both cell lines as early as 2 hr (FIG. 2). A ˜5-fold increase was seen in PC3AR by 2 hr with a peak of ˜8-fold by 16 hr. In PC3Neo, DR5 increased ˜2-fold by 2 hr after which no significant increase was noted. Also, DR4 levels increased in TRAIL-treated PC3AR cells, while PC3Neo cells did not show significant changes, suggesting that increased apoptotic response of PC3AR cells may be due to higher response of DR5 and DR4. Notably, DcR2 expression was significantly higher in PC3Neo cells compared to TRAIL-sensitive PC3AR cells (FIG. 2C). TRAIL treatment did not significantly alter DcR2 expression in either cell line. DcR1 expression was lower than DcR2 and was similar in both PC3Neo and PC3AR.

### Example 6

### Induction of Apoptosis Involves Activation of Caspase-8 and Truncation of Bid

Similar to TNFα, caspase-8 activation may be a prerequisite for the effects of TRAIL (Leverkus, M., et al., Cancer Res., 60, 553-559, 2000; Hao, C., et al., Cancer Res., 61, 1162-1170, 2001; Eggert, A., et al., Cancer Res., 61, 1314-1319, 2001; Lacour, S., et al., Cancer Res., 61, 1645-1651, 2001; Seol, D-W., et al., Cancer Res., 61, 1138-1143, 2001). In FIG. 3, lanes 1 correspond to untreated cells, lanes 2 correspond to Mifepristone treated cells, lanes 3 correspond to TRAIL treated cells, and lanes 4 correspond to cells treated with Mifepristone and TRAIL. Examination of the levels of caspase-8 in LNCaP cells treated with TRAIL (lanes 3) showed a decrease in the 57 KDa procaspase-8 protein as early as 2 hr, with concomitant appearance of two cleaved activated products of 46 KDa and 18 KDa (FIG. 3A). Treatment with both Mifepristone and/or TRAIL (lanes 4), increased the 18KDa activated product suggesting enhanced caspase-8 activity. Activation of procaspase-8 in LNCaP C4-2 (FIG. 3B) was similar to LNCaP indicating that activation of procaspase-8 is not responsible for differences in the response of the cells to TRAIL.

In both PC3Neo and PC3AR cells, TRAIL induced cleavage of procaspase-8 into the cleaved intermediate (p43 and p41) and mature (p18) products within 2 hr, with a greater increase in PC3AR than PC3Neo cells (not shown). Thus the p18 caspase-8 cleaved product increased in PC3AR by 2 hours and lasted at least 16 hours (with some decrease in intensity over the course of the experiment). In PC3Neo cells, the strong increase in activated caspase-8 seen at 2 hours was significantly reduced by 16 hours of TRAIL treatment (not shown). These results suggest that TRAIL induced robust and prolonged activation of caspase-8 in both PC3Neo and PC3AR, with more significant and long lasting effect in PC3AR cells.

Levels of tBid appear to correlate with the apoptic response. Bid is a 22 KDa BH3 domain only pro-apoptotic member of the BCl2 family, which is truncated into a 15 KDa protein (tBid) by activated caspase-8 (Li, H., et al., Cell 94, 491-501, 1998; Gross, A., et al., J. Biol. Chem., 274, 1156-1163, 1999). When LNCaP were treated with TRAIL individually (lanes 3), tBid was noted by 2 hr, although combination therapy with TRAIL and Mifepristone (lanes 4) resulted in more intense signal for tBid (FIG. 3A). tBid was higher in LNCaP C4-2 and was present till about 16 hr of treatment (FIG. 3B).

Treatment of PC3Neo and PC3AR cells with TRAIL also results in an increase in tBid expression, with significantly more induction seen in PC3AR cells than PC3Neo cells (not shown). Thus, tBid was detected up to 8 hr of treatment in PC3AR cells, while little tBid was present in PC3Neo cells by 4 hr of TRAIL treatment. As truncated Bid is translocated into mitochondria during apoptosis, cells were treated with TRAIL and mitochondrial fractions were isolated and analyzed for the presence of tBid. tBid levels in mitochondria of TRAIL treated PC3AR was significantly higher compared to PC3Neo (not shown). Thus, tBid was translocated into mitochondria within 2 hours of treatment with TRAIL, and continued throughout 8 hours of treatement. In contrast, the highest level of tBid in mitochondria in PC3Neo cells was seen by 2 hr, but the levels were significantly lower than that seen in PC3AR cells, further suggesting that TRAIL treated PC3AR are more apoptotic.

### Example 7

### Activation of Caspases 3, 9 and 7 by TRAIL

Activation of caspase-8 leads to either direct activation of procaspase-3 or its indirect activation via caspase-9. Alternatively, caspase-3 may be indirectly activated through the caspase-9 pathway. Activation of caspase-3 yields two cleaved products: an initial 17 KDa protein and a mature 12 KDa protein.

TRAIL treatment of LNCaP cells (lanes 3) yielded cleaved 17 KDa caspase-3 product by 2 hr that increased by 6 hr. However, treatment with both TRAIL and Mifepristone (lanes 4) for 6 hr were required for maximum activation of caspase-3, with the appearance of p12 product (FIG. 3C). Similar treatment of LNCaP C4-2 showed significantly increased p17 band by 2 hr (FIG. 3D), which increased significantly with time. In LNCaP C4-2, the p12 form appeared as early as 2 hr and continued throughout the experiment. These results indicate that caspase-3 activity was sustained and robust in LNCaP C4-2 compared to LNCaP.

Similarly, treatment of LNCaP cells with TRAIL activated caspase-7 by 20 hr, while pre-treatment of LNCaP cells with Mifepristone activated caspase-7 by 4 hr (FIG. 3C). In contrast, activated caspase-7 was noted in LNCaP C4-2 within 2 hr of treatment (FIG. 3D). These results demonstrate that LNCaP C4-2 are more sensitive to drugs compared to LNCaP, and caspase-3 activation precedes that of caspase-7.

As caspase-9 is a key protein in the formation of apoptosome, assays were performed to determine the activity of caspase-9. Caspase-9 activity was assayed using a colorimetric substrate, Ac-LEND-pNA with a kit from Chemicon International, Inc. (Temecula, Calif.). Cleavage of the C-terminal peptide bond by the enzyme released p-nitroaniline, which was measured at 405 nm. Pure recombinant human caspase-9 was utilized as a positive control. Treatment of cells with TRAIL and/or Mifepristone, induced caspase-9 activity (FIGS. 4A and B). In TRAIL-treated LNCaP and LNCaP C4-2 cells, pre-treatment with Mifepristone significantly increased caspase-9 function activity.

Similar results were found using PC3AR and PC3Neo cells. Upon treatment of cells with TRAIL, significant levels of cleaved caspase-9 were detected in PC3AR cells by 4 hr and lasted until 16 hr, whereas activated caspase-9 was not detected in PC3Neo. Cleaved caspase-3 (p17) was present in both cell lines as early as 2 hr of treatment, whereas mature p12 product was noted only in PC3AR, indicating significant activation of caspase-3 in these cells (not shown).

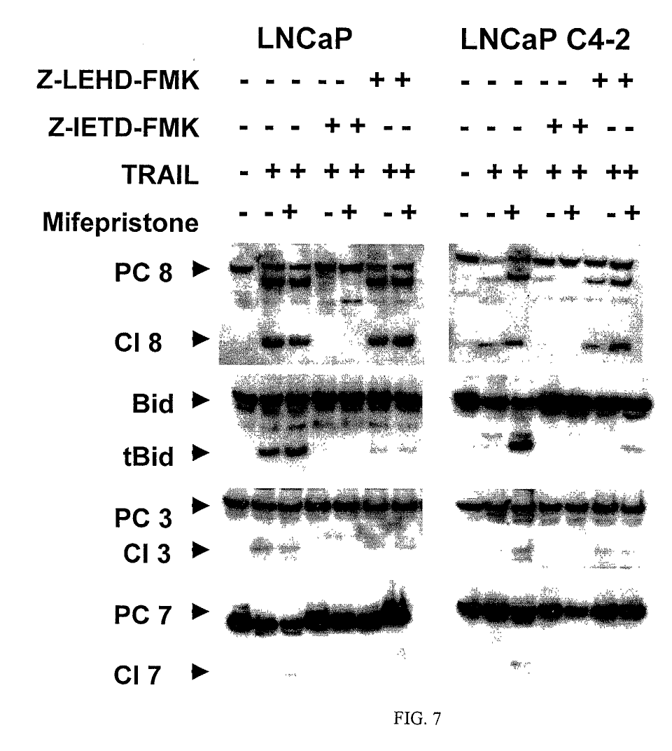

To further confirm the role of specific caspases in apoptotic response, inhibitors were utilized to specifically block caspases as described in Example 1. The caspase-8 inhibitor, Z-IETD-FMK, blocked activation of caspase-8 as noted by the absence of both intermediate and p18 cleaved products of caspase-8 in LNCaP and LNCaP C4-2 (FIG. 7). Notably, tBid was not present in these cells further confirming that caspase-8 is mainly responsible for truncation of Bid. Furthermore, caspase-3 and caspase-7 were not activated in cells with caspase-8 inhibitor. Inhibition of caspase-9 with the caspase-9 inhibitor, Z-LEHD-FMK, did not affect caspase-8 activity in response to treatment with TRAIL and/or Mifepristone. However, inhibition of caspase-9 activity significantly reduced the levels of tBid (FIG. 7), suggesting that in addition to caspase-8, caspase-9 affected truncation of Bid. Also, inhibition of caspase-9 reduced caspase-3 activity, although cleaved caspase-3 products were noted, probably due to direct activation of caspase-3 by caspase-8. Inhibition of caspase-9 blocked activation of caspase-7, indicating that caspase-9 is responsible for activation of caspase-7.

### Example 8

### Cytochrome C is Release in Response to Treatment by TRAIL

For separation of mitochondrial and cytosolic fractions, cells were trypsinized, centrifuged at 600 g for 10 min at 4° C., washed twice in ice-cold PBS and re-suspended in buffer A (20 mM HEPES-KOH, pH 7.2, 10 mM KCl, 1.5 mM MgC1₂, 1 mM sodium-EDTA, 1 mM sodium-EGTA, 250 mM sucrose and protease inhibitor cocktail). Cells were homogenized on ice with a glass Dounce homogenizer (30 strokes) and were centrifuged at 750 g for 5 min at 4° C. The supernatant was centrifuged for 15 min at 10,000 g at 4° C. to pellet mitochondria, which was washed and resuspended in lysis buffer. The supernatant was centrifuged at 100,000 g for 60 min at 4° C. and S-100 cytosol was collected.

Treatment of LNCaP (panel A) and LNCaP (panel B) C4-2 cells with TRAIL increased cytochrome c levels in cytosol within 15 min, with the increase in cytosolic cytochrome c being highly significant by 30 min (FIG. 5). Cytochrome c levels induced by TRAIL were significantly higher in LNCaP C4-2 cytosol compared to LNCaP. As LNCaP C4-2 were responsive to TRAIL, while LNCaP were resistant (FIG. 1), increased levels of cytochrome c in LNCaP C4-2 suggest that mitochondria from these cells may be more sensitive to the effects of TRAIL. Levels of cytochrome c were higher in cells treated with both TRAIL and Mifepristone, and similar to results with TRAIL-treated cells, the cytosolic cytochrome c levels in LNCaP C4-2 were higher than that in LNCaP.

### Example 9

### The Role of NFκB in TRAIL-Induced Apoptosis

cFLIP (also known as Flame, CASH, Clarp, MRIT, Casper, I-Flice, Usurpin), NFκB and XIAP influence apoptotic mechanisms. As cFLIP associates with procaspase-8 and competes for binding to FADD, increased levels of cFLIP affects caspase-8 function. Therefore, to determine whether cFLIP is responsible for differential action of caspase-8 pathway by TRAIL in prostate cells, cFLIP levels in PC3Neo and PC3AR were examined. Our analysis demonstrated no significant differences in the expression of cFLIP in the two cell lines (not shown), suggesting that cFLIP may not be responsible for the differences in apoptotic response.

Examination of NFκB and XIAP, which are known to affect apoptosis, revealed that PC3Neo expressed higher levels of NFκB and XIAP compared to PC3AR (not shown). Treatment with TRAIL increased the expression of NFκB levels in both PC3Neo and PC3AR cell lines. As the basal levels of NFκB were low in TRAIL-sensitive PC3AR, the increase in expression of NFκB in TRAIL-treated cells was highly significant in PC3AR cells. Similar analysis if XIAP revealed that its basal levels were higher in PC3Neo, however, treatment of PC3Neo and PC3AR with TRAIL did not alter XIAP levels significantly (not shown). These results suggested that resistance of androgen-insensitive PC3Neo to TRAIL, may be due to higher levels of NFκB and XIAP, two key proteins influencing cell survival.

To examine the role of NFκB in altered response to TRAIL, its function was blocked by over expressing mutated IκB protein (Kanegae, Y., et al., Nature 392, 611-614, 1998). Cells (6000 cells/well) were plated in 96-well plates and infected with adenoviral construct, pAxCAIκB-M at the rate of 100 viral particles/cell (the construct was provided by Dr. I. M. Verma, Salk Institute for Biological Studies, La Jolla, Calif.). The construct is a dominant negative mutant of IκB in which the inducible phosphorylation sites (Ser32 and Ser36) and constitutive phosphorylation site at its carboxy terminus were substituted with alanine (Kanegea, Y., et al., Nature 392, 611-614, 1998). Due to defects in phosphorylation, IκB stays bound to NFκB affecting its nuclear translocation and function. Cells were incubated at 37° C. for 3 hr with the virus before treatment with TRAIL (400 ng/ml) for 20 hr.

Infection with the IκBM construct increased apoptosis in both cell lines, although the response was higher in PC3Neo (FIG. 6A), confirming the protective role of NFκB to TRAIL in these cells. Increased expression of Iκβ protein in infected cells was confirmed by western analysis (FIG. 6B). Interestingly, increased expression of Iκβ coincided with decreased expression of XIAP in PC3Neo (FIG. 6B), while a similar decrease was not noted in PC3AR cells. Thus, increased levels of NFκB and XIAP in PC3Neo cells may be responsible for reduced response to TRAIL.

The ability of NFκB to affect cellular functions related to apoptosis in prostate cells was examined. Expression of IκB mutant protein resulted in further increase of cleaved intermediate (p43 and p41) and mature (p18) caspase-8 products in both PC3Neo and PC3AR cells, indicating that activation of caspase-8 is inhibited by endogenous NFκB (FIG. 6C). The fold induction was higher in PC3Neo cells, suggesting that higher levels of NFκB in PC3Neo limit the response of the cells to TRAIL.

Furthermore, truncation of Bid increased further when NFκB function was blocked. Increase in signal for tBid was similar to the pattern seen for caspase-8 in both PC3AR and PC3Neo (FIG. 6C). These results indicate that NFκB suppresses the response of the cells to TRAIL. Still, blocking NFκB function did not increase the apoptotic response of PC3Neo to the level of PC3AR, showing that in addition to NFκB other proteins, such as IAPs, may be influencing this response.

### Example 10

### The Reduced Response of LNCaP to TRAIL is Not Mediated Through Akt

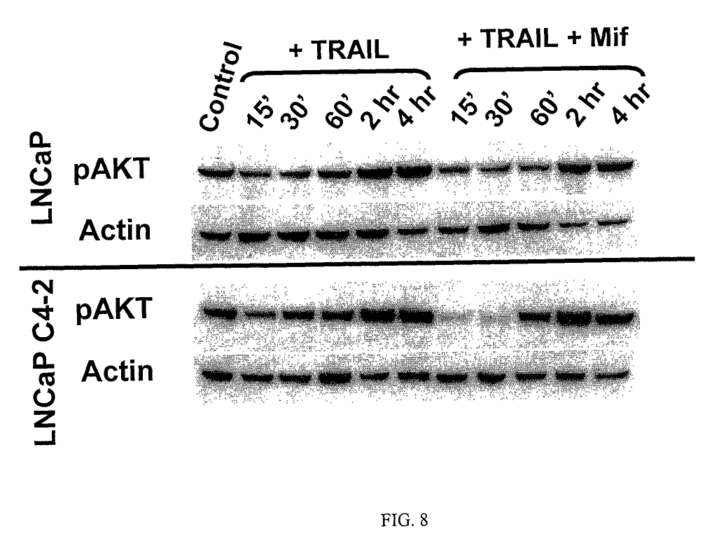

Recent reports suggested that the lack of response of LNCaP to TRAIL may be due to increased levels of Akt, a protein implicated in cell survival pathways (Nesterov, A., et al., J. Biol. Chem., 276, 10767-10774, 2001). To determine whether Akt accounted for response to TRAIL, the levels of Akt in LNCaP and LNCaP C4-2 cells treated with TRAIL were compared. Treatment of cells with TRAIL for 15 min slightly decreased phosphorylated Akt in both LNCaP and LNCaP C4-2 (FIG. 8), with greater effects seen for TRAIL+Mifepristone than for either agent alone. Levels of Akt returned to control levels by 60 minutes, and eventually exceeded levels seen in the controls. It appears, therefore, that the decreased response of LNCaP to TRAIL is not due to differences in the levels of phosphorylated Akt.

Thus, the present invention describes the use of TRAIL and an antiprogestin such as Mifepristone for the treatment of prostate cancer. The invention relies on the discovery that Mifepristone enhances aspects of the TRAIL pathway of apoptosis. Thus, treatment of cells which are resistant to the apoptic effects of TRAIL with Mifepristone leads to cellular responses, such as activation of caspases 8 and 9 (and subsequently caspases 3 and 7) which result in apoptosis.

The invention has been described in detail with particular reference to preferred embodiments thereof, but it will be understood that variations and modifications can be effected within the spirit and scope of the invention. References cited herein are incorporated in their entirety by reference unless otherwise noted.

What is claimed is:

1. A method for inducing cell death in cancer cells, the method comprising treating cancer cells with an effective amount of TRAIL sufficient to induce apoptosis in at least a portion of the treated cancer cells.

2. A method for inducing cell death in cancer cells, the method comprising treating cancer cells with an effective amount of TRAIL and an effective amount of an antiprogestin sufficient to induce apoptosis in at least a portion of the treated cancer cells.

3. The method of claim 2, wherein the antiprogestin comprises Mifepristone.

4. A method for treating cancer by inducing cell death in cancer cells, the method comprising treating cancer cells with a pharmaceutical composition comprising an effective amount of TRAIL and an effective amount of Mifepristone sufficient to induce apoptosis in at least a portion of the treated cancer cells.

5. The method of claim 4, wherein the cancer cells are treated with Mifepristone prior to being treated with TRAIL.

6. The method of claim 4, wherein the cancer cells are treated with Mifepristone and TRAIL concurrently.

7. The method of claim 4, wherein the dose of TRAIL in said pharmaceutical composition results in a local concentration of TRAIL at the tumor which ranges from 1 to 1,000 ng/ml.

8. The method of claim 4, wherein the dose of TRAIL in said pharmaceutical composition results in a local concentration of TRAIL at the tumor which ranges from 200 to 600 ng/ml.

9. The method of claim 4, wherein the dose of TRAIL in said pharmaceutical composition results in a local concentration of TRAIL at the tumor which ranges from 350 to 450 ng/ml.

10. The method of claim 4, wherein the dose of Mifepristone in said pharmaceutical composition results in a local concentration of Mifepristone at the tumor which ranges from 1 to 1,000 μM.

11. The method of claim 4, wherein the dose of Mifepristone in said pharmaceutical composition results in a local concentration of Mifepristone at the tumor which ranges from 1 to 100 μM.

12. The method of claim 4, wherein the dose of Mifepristone in said pharmaceutical composition results in a local concentration of Mifepristone at the tumor which ranges from 5 to 20 μM.

13. The method of claim 4, wherein said cancer cells comprise prostate cancer cells.

14. The method of claim 13, wherein said prostate cancer cells comprise androgen responsive cells.

15. The method of claim 13, wherein said prostate cancer cells comprise cells which do not respond to androgen.

16. The method of claim 4, wherein the treatment of cancer cells with TRAIL and Mifepristone is associated with an increase in at least one death receptor in at least a portion of the treated cells.

17. The method of claim 16, further comprising an increase in the death receptor DR4 and/or DR5.

18. The method of claim 4, wherein the treatment of cancer cells with TRAIL and Mifepristone is associated with an increase in activated caspase enzymes.

19. The method of claim 18, wherein said activated caspases comprise caspase-8, caspase-7, caspase-9, or caspase-3.

20. The method of claim 4, wherein the treatment of cancer cells with TRAIL and Mifepristone is associated with an increase in truncated BID protein (tBid) in at least a portion of the treated cells.

21. The method of claim 4, wherein the treatment of cancer cells with TRAIL and Mifepristone is associated with a reduction in mitochondrial function.

22. The method of claim 4, wherein the treatment of cancer cells with TRAIL and Mifepristone results in an increase in apoptosome formation in at least a portion of the treated cells.

23. The method of claim 4, further comprising treating said cancer cells with a compound which reduces the concentration of active NFκB in said cells.

24. The method of claim 23, further comprising treating said cancer cells with IkB or an analogue thereof, wherein said analogue comprises a polypeptide which prevents activation of NFκB.

25. The method of claim 4, wherein the manner of treatment comprises intravenous injection of said pharmaceutical composition.

26. The method of claim 4, in combination with other means of treatment such as surgery, chemotherapy, or radiation therapy.

27. A composition for treating cancer by inducing cell death in cancer cells comprising an effective amount of TRAIL in a pharmaceutical carrier, wherein an effective amount comprises sufficient TRAIL to induce apoptosis in at least a portion of said cancer cells exposed to said composition.

28. A composition for treating cancer by inducing cell death in cancer cells comprising an effective amount of TRAIL and an antiprogestin in a pharmaceutical carrier, wherein an effective amount comprises sufficient TRAIL and antiprogestin to induce apoptosis in at least a portion of said cancer cells exposed to said composition.

29. The composition of claim 28, wherein the antiprogestin comprises Mifepristone.

30. A composition for treating cancer by inducing cell death in cancer cells comprising an effective amount of TRAIL and Mifepristone in a pharmaceutical carrier, wherein an effective amount comprises sufficient TRAIL and Mifepristone to induce apoptosis in at least a portion of said cancer cells exposed to said composition.

31. The composition of claim 30, wherein said Mifepristone and said TRAIL are packaged in such a manner that said Mifepristone is at least partially released for application to the cancer prior to the release of said TRAIL.

32. The composition of claim 30, wherein said Mifepristone and said TRAIL are packaged in such a manner so as to be released substantially simultaneously.

33. The composition of claim 30, wherein the dose of TRAIL results in a local concentration of TRAIL at the tumor which ranges from 1 to 1,000 ng/ml.

34. The composition of claim 30, wherein the dose of TRAIL results in a local concentration of TRAIL at the tumor which ranges from 200 to 600 ng/ml.

35. The composition of claim 30, wherein the dose of TRAIL results in a local concentration of TRAIL at the tumor which ranges from 350 to 450 ng/ml.

36. The composition of claim 30, wherein the dose of Mifepristone results in a local concentration of Mifepristone at the tumor which ranges from 1 to 1,000 μM.

37. The composition of claim 30, wherein the dose of Mifepristone results in a local concentration of Mifepristone at the tumor which ranges from 1 to 100 μM.

38. The composition of claim 30, wherein the dose of Mifepristone results in a local concentration of Mifepristone at the tumor which ranges from 5 to 20 μM.

39. The composition of claim 30, wherein said cancer cells comprise prostate cancer cells.

40. The composition of claim 39, wherein said prostate cancer cells comprise androgen responsive cells.

41. The composition of claim 39, wherein said prostate cancer cells comprise cells which do not respond to androgen.

42. A kit for pharmaceutical treatment of cancer comprising:
(a) a pharmacologically effective amount of TRAIL packaged in a sterile container;
(b) a pharmacologically effective amount of an antiprogestin packaged in a sterile container;
(c) at least one aliquot of a pharmaceutical carrier; and
(d) instructions for application of said TRAIL and said antiprogestin to a patient having cancer.

43. The kit of claim 42, wherein said antiprogestin comprises Mifepristone.

44. The kit of claim 42, wherein said cancer comprises prostate cancer.
